(19) United States
(12) Patent Application Publication
(10) Pub. No.: US 2002/0115688 A1
(43) Pub. Date: Aug. 22, 2002

(54) Screen for glutamate reuptake inhibitors, stimulators, and modulators

(76) Inventors: Philip M. Beart, Ivanhoe (AU); Ross D. O'Shea, Blackburn South (AU); Karina Aprico, Forest Hill (AU); Andrew J. Lawrence, Brighton (AU); Maria-Luisa Maccecchini, West Chester, PA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 09/944,954

(22) Filed: Sep. 1, 2001

Related U.S. Application Data
(60) Provisional application No. 60/229,952, filed on Sep. 1, 2000.
(60) Provisional application No. 60/230,159, filed on Sep. 1, 2000.

(51) Int. Cl. A61K 31/47; G01N 33/53

(52) U.S. Cl. 514/312; 435/7.1

(57) ABSTRACT

Disclosed is a method for identifying compounds that bind to or modulate a glutamate transporter. The disclosed method is useful for identifying compounds that can inhibit, stimulate, or modulate the activity of the glutamate transporter and thus affect glutamate reuptake. The method is a screening technique where compounds known to bind to glutamate receptors (for example, glutamate receptor ligands, including many agonists, and antagonists) are bound to a glutamate transporter and compounds are screened to identify those that can alter the binding of the glutamate receptor-binding compounds. Compounds shown to alter the binding of the receptor compounds from glutamate transporters in the disclosed assay can have a variety of effects on glutamate transporter activity including activation or inhibition. These compounds are expected to affect or interfere with glutamate reuptake by the glutamate transporter and thus can be used to modulate, stimulate, or inhibit glutamate reuptake. Such compounds are useful to treat various neurological diseases and conditions involving glutamate transporter and glutamate receptor activation. One of the compounds is (2S,4R)-4-methylglutamate or [³H]-(2S,4R)-4-methylglutamate. For example, excess extracellular glutamate is a cause of excessive activation of glutamate receptors. Stimulating glutamate reuptake by glutamate transporters can ameliorate excessive activation of glutamate receptors by reducing the extracellular glutamate concentration. Prodrug forms of transporter compounds are preferred for use as drugs.

### CROSS-REFERENCE To RELATED APPLICATIONS

Priority is claimed to U.S. Provisional application Ser. Nos. 60/229,952 and 60/230,159, both filed Sep. 1, 2000, the teachings of which are incorporated herein.

### BACKGROUND OF THE INVENTION

This invention generally relates to glutamate transporters and more specifically to methods for identifying inhibitors, stimulators, and modulators of glutamate transporters.

During the past twenty-five years, a revolution in understanding the basic structure and chemistry of the synaptic interconnections of neural tissues has taken place, which has yielded knowledge relevant to the treatment of neural tissue damage and disorders. The studies have centered around an understanding of the properties of the neurochemical transmitters released from presynaptic membranes and, most importantly, the postsynaptic receptors for these transmitters. The nicotinic acetylcholine and γ-aminobutyric acid (GABA) receptors have been characterized and found to be under the control of allosteric modulators. During the past ten years, a great deal of attention has been directed to the excitatory amino acids (EAAs), principally glutamic acid (the primary excitatory neurotransmitter) and aspartic acid, and their receptors since these amino acids mediate the fast excitatory transmission in the mammalian central nervous system. Thus, glutamic acid can bring about changes in the postsynaptic neuron that reflect the strength of the incoming neural signals.

Two major classes of EAA receptors are distinguished: ionotropic and metabotropic. The ionotropic receptors contain ligand-gated ion channels and mediate ion fluxes for signaling, while the metabotropic receptors use G-proteins for signaling. Further sub-classification of the ionotropic EAA glutamate receptors is based upon the agonists (stimulating agents) other than glutamic and aspartic acid that selectively activate the receptors. Presently, it is believed that there are three major subtypes of ionotropic glutamate receptors based on binding at defined concentrations: 1) a receptor responsive to N-methyl-D-aspartate (NMDA); 2) a receptor responsive to α-amino-3-hydroxy-5-methyl-4-isoxazolepropionic acid (AMPA); and 3) a receptor responsive to KA. The NMDA receptor controls the flow of both divalent (Ca⁺⁺) and monovalent (Na⁺, K⁺) ions into the postsynaptic neural cell although it is the Ca⁺⁺ flux which is of the greatest interest. The AMPA and KA receptors also regulate the flow into postsynaptic cells of monovalent K⁺ and Na⁺and occasionally divalent Ca⁺⁺. The metabotropic EAA receptors are divided into three sub-groups that are unrelated to ionotropic receptors, and are coupled via G-proteins to intracellular second messengers. These metabotropic EAA receptors are classified based on receptor homology and second messenger linkages, and there continue to be reports of novel metabotropic EAA receptors.

EAA receptors have been implicated during development in specifying neuronal architecture and synaptic connectivity and may be involved in experience-dependent synaptic modifications. These receptors have also drawn interest since they appear to be involved in a broad spectrum of CNS disorders. For example, during brain ischemia caused by stroke or traumatic injury, excessive amounts of the EAA glutamic acid are released from damaged or oxygen deprived neurons. Binding of this excess glutamic acid to the postsynaptic glutamate receptors opens their ligand-gated ion channels, thereby allowing an ion influx which in turn activates a biochemical cascade resulting in protein, nucleic acid and lipid degradation and cell death. This phenomenon, known as excitotoxicity, is also thought to be responsible for the neurological damage associated with other disorders ranging from hypoglycemia, ischemia, and epilepsy to chronic neurodegeneration in Huntington's, Parkinson's, and Alzheimer's diseases.

L-Glutamate (Glu) is the major excitatory neurotransmitter in the central nervous system (CNS) and acts at distinct subtypes of ionotropic (iGluRs) and metabotropic (mGluRs) receptors. Excessive activation of iGluRs and group I mGluRs may result in neuronal death. Many neurodegenerative conditions, including Parkinson's disease, Alzheimer's disease, cerebral ischaemia, epilepsy, Huntington's chorea and amyotrophic lateral sclerosis (ALS), have been linked to disturbed Glu homeostasis (Lipton and Rosenberg, New Eng. J Med., 330: 613-622 (1994); Gegelashvili and Schousboe, Mol. Pharmacol., 52: 6-15 (1997); Robinson and Dowd, Adv. Pharmacol,., 37: 69-115 (1997)). Extracellular Glu concentrations are maintained within physiological levels exclusively by Glu transporters (GluTs; also known as amino acid transporters), since no extracellular enzymes exist for the breakdown of Glu (Robinson and Dowd, 1997). Thus, GluTs are responsible for the high-affinity uptake of extracellular Glu, and permit normal excitatory transmission as well as protecting against excitotoxicity (Robinson and Dowd, 1997). The abundance and function of GluTs is therefore clearly relevant to neurodegenerative diseases. Overall, Glu interacts with various proteomic binding sites, be they receptors or membrane transporters, and on the basis of pharmacological studies using conformationally restricted molecules it is clear that these interactions involve multiple conformations of Glu. A number of compounds acting on the ionotropic EAA receptors have been described.

Since 1992, molecular cloning and molecular biological studies have identified five subtypes of GluT (nomenclature in human EAAT1-5) demonstrating discrete cellular and regional localizations as well as distinct molecular and pharmacological characteristics. GLAST (the rodent homologue of the human EAAT1) is predominantly located in cerebellar Bergmann glia, but is also present in glial cells throughout the CNS and in a small number of neurones under certain conditions. GLT1 (EAAT2) is almost exclusively glial and is widespread and abundant throughout the forebrain, cerebellum and spinal cord. The transporters EAAC1 (EAAT3) and EAAT4 are found predominantly in neurones, with EAAC1 being abundantly expressed throughout the brain and in spinal cord, and EAAT4 unique to Purkinje cells of the cerebellum. EAAT5 is expressed predominantly in the retina, but its cellular specificity is yet to be determined. The majority of synapses in the CNS are in close apposition with glial cells, and glial GluTs appear to be responsible for most Glu transport in the CNS.

(2S,4R)-4-Methyglutamate (4MG) is a desensitizer at the low affinity kainate receptor (U.S. Pat. No. 5,731,348, Gu et al., J. Medicinal Chem. 38:2518 (1995), Zhou et al., J. Pharmacology Exper. Therapeutics 280:422-427 (1997)), but also acts as a non-transportable blocker of GLT1 (Kₘ=3.4 μM) and a competitive inhibitor of GLAST (Kₘ=54 μM)(Vandenberg et al., Mol. Pharmacol., 51: 809-815 (1997)). Carroll et al., Neurosci. Lett., 255: 71-74 (1998), and Toms et al., Neuropharmacology, 36: 1483-1488 (1997), describe use of [³H]-4MG to study the distribution and properties of low affinity kainate receptors. In rabbit brain membranes, Toms et al. (1997) found two binding sites for [³H]-4MG. In contrast, Carroll et al. (1998) demonstrated that [³H]-4MG bound only to a single population of sites (low affinity kainate receptors) in sections of Macaca fascicularis monkey brain. In both studies, the pharmacological profile of binding clearly establishes that all binding is to Glu receptors.

Comparatively little is known about the various glutamate transporters. A principal reason for this lack of knowledge is that few compounds are known which selectively bind to individual subtypes of glutamate transporters, and also because the interpretation of the pharmacology of these compounds is generally complicated by concurrent direct actions at EAA receptors.

Many compounds are known to bind to EAATs and inhibit transporter function. Inhibitors of EAATs fall into two major classes that differ in their mode of action: non-transportable blockers and competitive substrates. Most available inhibitors are competitive substrates, which are generally transported at a slower rate than Glu and may displace cytoplasmic Glu through heteroexchange, leading to further extracellular accumulation of Glu.

In addition to direct actions on transport, EAATs are subject to complex modulation by a variety of compounds, including glutamate receptor agonists, zinc, arachidonic acid, cyclic AMP and nitric oxide, which appear to effect the expression, mobilization and function of EAATs.

Chemists and pharmacologists have attempted to understand the critical aspects of shape, pharmacophore position and pharmacophore type that are important for positive or negative modulation of the transporters. Generally random screening and hit or miss synthesis and testing were used to find new modulators for the transporters. It is likely that each glutamic acid transporter subtype, such as GLAST and GLT1, will have its own requirements and pharmacophore positions. The optimal way to design new non-transportable blockers and competitive substrates is to have a non-transportable blocker and/or competitive substrate model for each transporter subtype that contains the specific shape and pharmacophore positions and then to use this model to link the pharmacophores into novel molecules.

Much of what has been learned about glutamate receptors has been made possible by the discovery of compounds which block one or another action of the various modulatory agents. The approach of using blocking agents to map pathways has a long history in biochemical and biophysical research and very often these blocking agents have been discovered to be useful therapeutic agents. A similar approach, although applicable to glutamate transporters, cannot be used in the absence of compounds which selectively bind to a particular glutamate transporter subtype. It would be useful to have a screen for compounds that bind to or affect particular subtypes of glutamate transporters.

It is therefore an object of the present invention to provide a method of identifying compounds that inhibit, stimulate, or modulate glutamate reuptake.

It is a further object of the present invention to provide compounds which selectively inhibit, stimulate, modulate, or regulate glutamate reuptake.

### BRIEF SUMMARY OF THE INVENTION

Disclosed is a method for identifying compounds that bind to glutamate transporters. The disclosed method is useful for identifying compounds that can inhibit, stimulate, or modulate the activity of the glutamate transporter and thus affect glutamate reuptake. The method is a screening technique where compounds known to bind to glutamate receptors (for example, glutamate receptor ligands, agonists, and antagonists) are bound to a glutamate transporter and compounds are screened to identify those that can alter the binding of the glutamate receptor-binding compounds. Many glutamate receptor-binding compounds are known. It has been discovered that many such compounds also bind glutamate transporters and thus can be used in the disclosed screening method. Glutamate receptor-binding compounds useful in the disclosed method are referred to herein as receptor compounds. Compounds identified in the assay as compounds that can alter the binding of a receptor compound to a transporter are referred to herein as transporter compounds.

Transporter compounds—compounds shown to alter the binding of receptor compounds to glutamate transporters in the disclosed assay—can have a variety of effects on glutamate transporter activity including activation or inhibition. These compounds are expected to affect or interfere with glutamate reuptake by the glutamate transporter and thus can be used to modulate, stimulate, or inhibit glutamate reuptake. Such compounds are useful to treat various neurological diseases and conditions involving glutamate transporter and glutamate receptor activation. One of the compounds is (2S,4R)-4-methylglutamate or [³H]-(2S,4R)-4-methylglutamate. For example, excess extracellular glutamate is a cause of excessive activation of glutamate receptors. Stimulating glutamate reuptake by glutamate transporters can ameliorate excessive activation of glutamate receptors by reducing the extracellular glutamate concentration. Prodrug forms of transporter compounds are preferred for use as drugs.

### BRIEF DESCRIPTION OF THE DRAWINGS

FIG. 1 is a graph of association of [³H]-4MG to homogenized brain tissue (in percent specific binding) versus time (in minutes).

FIG. 2 is a graph of dissociation of [³H]-4MG to homogenized brain tissue (in percent specific binding) versus time (in minutes).

FIG. 3 is a graph of inhibition of binding of [³H]-4MG to homogenized brain tissue (in percent specific binding) versus concentration of various transporter compounds in molar units.

FIG. 4 is a graph of dissociation of [³H]-4MG to homogenized brain tissue (in percent specific binding) versus concentration of various transporter compounds in molar units.

FIG. 5 is a graph of specific binding (in percent control) of [³H]-D-aspartate and [³H]-4MG in the presence of various transporter and receptor compounds.

FIGS. 6A-6I are diagrams of the structures of examples of transporter compounds.

### DETAILED DESCRIPTION OF THE INVENTION

It has been discovered that ligands, agonists, and antagonists of glutamate receptors can bind to glutamate transporters. The binding of these compounds to glutamate transporters can be used in assays to identify other compounds that bind to glutamate transporters. Such compounds can bind to glutamate transporters and affect extracellular glutamate levels by affecting transporter activity. The disclosed compounds can have a variety of effects on glutamate transporter activity including activation or inhibition.

The prototype compound, 4-methylglutamate (4MG), is known to bind low affinity kainate receptors. It was discovered that, in the presence of near-physiological concentrations of the sodium ion, 4MG can bind selectively to glutamate transporters having characteristics of the glial glutamate transporters GLAST and GLT1. It was realized that many of the members of the class of compounds that are ligands of glutamate receptors, including many agonists and antagonists, will have similar properties. Thus, the disclosed method makes use of compounds that are ligands, agonists, or antagonists of glutamate receptors in a method to identify compounds that bind glutamate transporters. Such compounds are referred to herein as receptor compounds.

4-Methylglutamate is known to bind low affinity kainate receptors. It was discovered that 4MG can bind selectively to the glutamate transporters GLAST and GLT1. It was realized that many members of the class of compounds that are ligands of glutamate receptors, including many agonists, or antagonists, will have similar properties. This realization makes possible the disclosed method, and the resulting identification of compounds useful for treating diseases and conditions involving excitatory amino acids.

Labeled forms of the disclosed receptor compounds can also be used to selectively label all or a subset of the total population of GluTs. Additionally, labeled receptor compounds can be used for autoradiographic studies allowing the visualization of GluTs in slices of tissue. Any kind and type of tissue or membrane preparation can be used with the disclosed method. For example, fresh tissues, frozen tissues, fresh membranes, frozen membranes, commercially available membranes, gliomas, cell lines, and artificial membranes.

Glossary of Terms

The term “activity” as used herein in reference to a glutamate receptor refers to the flow of cations through the receptor. Increased activity means increased flow. Decreased activity means reduced flow. The term “activity” as used herein in reference to a glutamate transporter refers to the transport of excitatory amino acids by the transporter. Increased activity means increased transport. Decreased activity means decreased transport.

The term “activation” as used herein in reference to a glutamate receptor refers to an increase in the flow of cations through the receptor. The term “activation” as used herein in reference to a glutamate transporter refers to increased transport of excitatory amino acids by the transporter. The term “excessive activation” used in reference to a glutamate receptor refers to an activation resulting in the opening of an ion channel for a prolonged period of time so that there is an excessive ion flux through the channel, which results in substantial damages to the cell including cell death.

The term “ligand” as used herein means any compound which binds to a glutamate receptor, and includes but is not limited to agonists, antagonists and partial agonists.

The term “agonist” as used herein means any compound which increases the activity of a glutamate receptor, and which has not been observed to decrease the activity of the same receptor.

The term “antagonist” as used herein means any compound which decreases the activity of a glutamate receptor, and which has not been observed to increase the activity of the same receptor.

The term “partial agonist” as used herein means a compound which modulates the activity of a glutamate receptor or transporter depending on the presence or absence of the principal site modulator(s). In the absence of the principal site modulator(s), a partial agonist increases the activity of the glutamate receptor or transporter but at a lower level than achieved by the principal site modulator(s). A partial agonist partially activates the receptor or transporter. In the presence of the principal site modulator(s), a partial agonist decreases the activity of a glutamate receptor or transporter below the activity normally achieved by the principal site modulator(s).

The term “principal site ligand” as used herein refers to known endogenous ligands binding to a site.

The term “glutamic acid” as used herein means the amino acid L-glutamic acid (“Glu”).

The term “neuropsychopharmacological disorder” as used herein means a disorder resulting from, or associated with, a reduced or excessive flux of ions through a glutamate receptor ligand-gated cation channel or other effects of EAAT inhibition including impairment of intermediary metabolism involving neurons and glia, and includes cognitive, learning, and memory deficits, chemical toxicity (including substance tolerance and addiction), excitotoxicity, neurodegenerative disorder (such as Huntington's disease, Parkinson's disease, amyotrophic lateral sclerosis and Alzheimer's disease), post-stroke sequelae, epilepsy, seizures, mood disorders (such as bipolar disorder, dysthymia, anxiety, and seasonal effective disorder), and depression. Neurodegenerative disorders can result from dysfunction or malfunction of the receptor and/or transporter. As used herein, this term includes pain.

The term “NMDA receptor” as used herein means a presynaptic or postsynaptic receptor which is stimulated, at a minimum, by the EAA glutamic acid as well as by NMDA, but is not stimulated by AMPA or kainic acid. It is a ligand-gated receptor.

The term “AMPA receptor” as used herein means a presynaptic or postsynaptic receptor which is stimulated by the EAAs glutamic acid as well as by AMPA, but is not stimulated by NMDA and only minimally and at high concentrations by kainic acid. It is a ligand-gated receptor.

The term “KA receptor” as used herein means a presynaptic or postsynaptic receptor which is stimulated, at a minimum, by the EAA glutamic acid as well as by KA, but is not stimulated by NMDA. It is a ligand-gated receptor.

The term “metabotropic glutamate receptor” as used herein refers to a non-ionotropic, presynaptic or postsynaptic receptor which is stimulated, at a minimum, by the EAA glutamic acid, resulting in alterations in G-protein-linked second messenger signaling.

The term “potency” as used herein refers to the molar concentration at which a specified effect on a receptor channel or transporter activity is observed. Specifically, potency for a compound exhibiting antagonistic effect is presented as the IC₅₀ value, which is the concentration at which inhibition of activity is 50% of the maximum inhibition achievable. Lower values indicate higher potency. Potency for a compound exhibiting agonistic effect is presented as the EC₅₀ value, which is the concentration at which enhancement of activity is 50% that of the maximum enhancement achievable. Lower values indicate higher potency.

The term “efficacious” as used herein refers to a comparison of the maximum increase or decrease in activity achieved by a particular compound with maximum increase or decrease in activity achieved by a principal site ligand. Efficacy refers to magnitude of a specified effect.

The term “pharmacophore” as used herein means an atom or group of atoms that electrostatically or through hydrogen bonds interacts directly with the receptor or transporter protein.

The term “specifically binds” as used herein means a compound binding to a receptor or transporter with an affinity at least three times as great as a compound which binds to multiple sites, receptors, or transporters.

The term “prodrug” as used herein means a compound that is converted into a bioactive form in an animal body. The prodrug itself may or may not have a bioactivity.

Materials

The disclosed method can be carried out, and the identified compounds can be used, by employing the following materials as described below and elsewhere herein.

A. Receptor Compounds

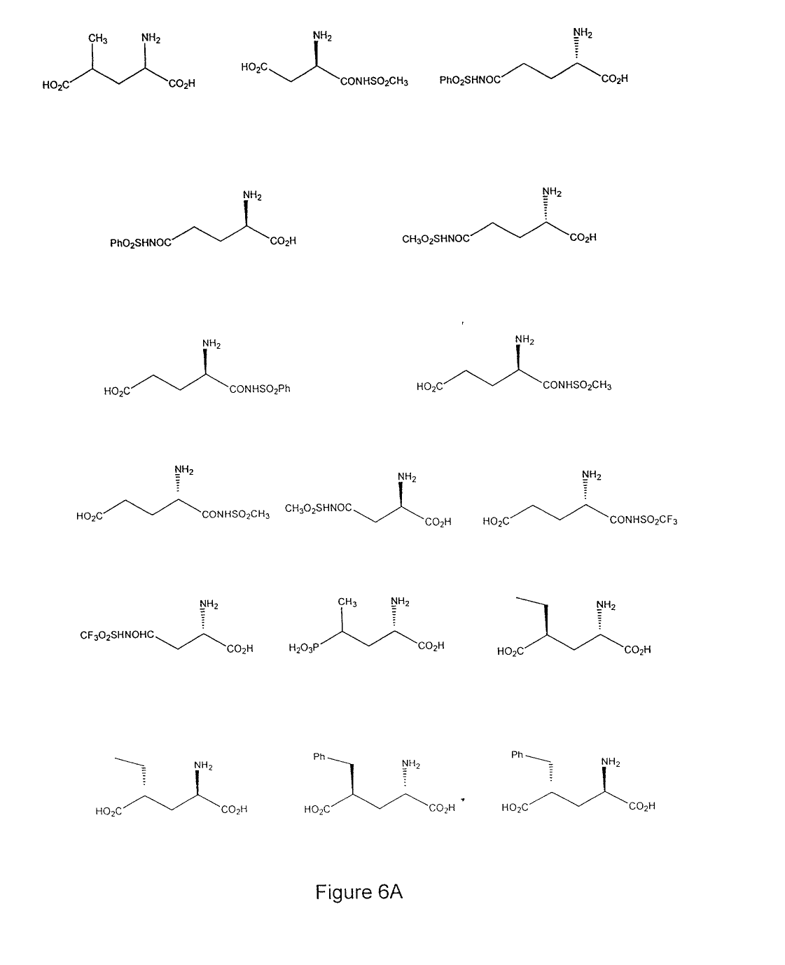

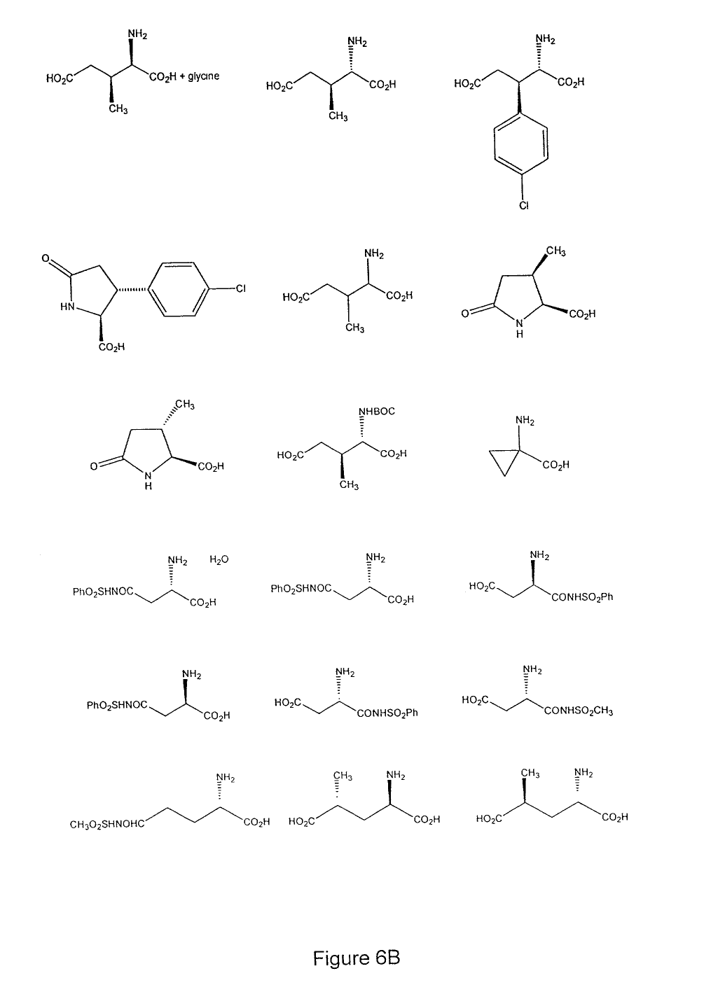

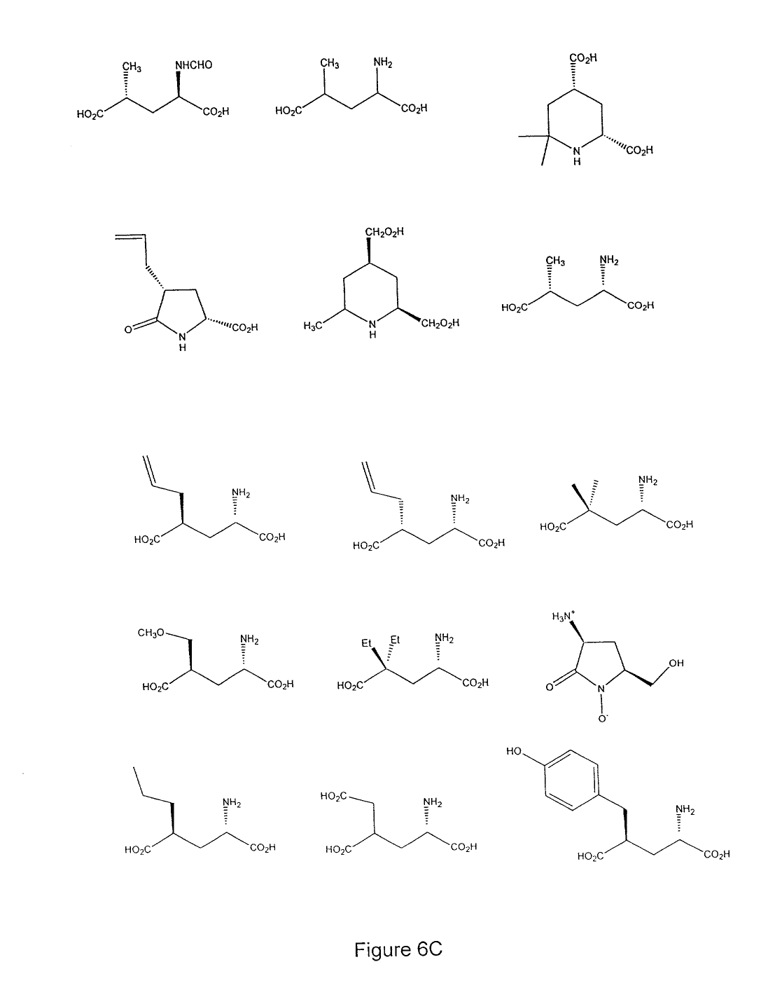

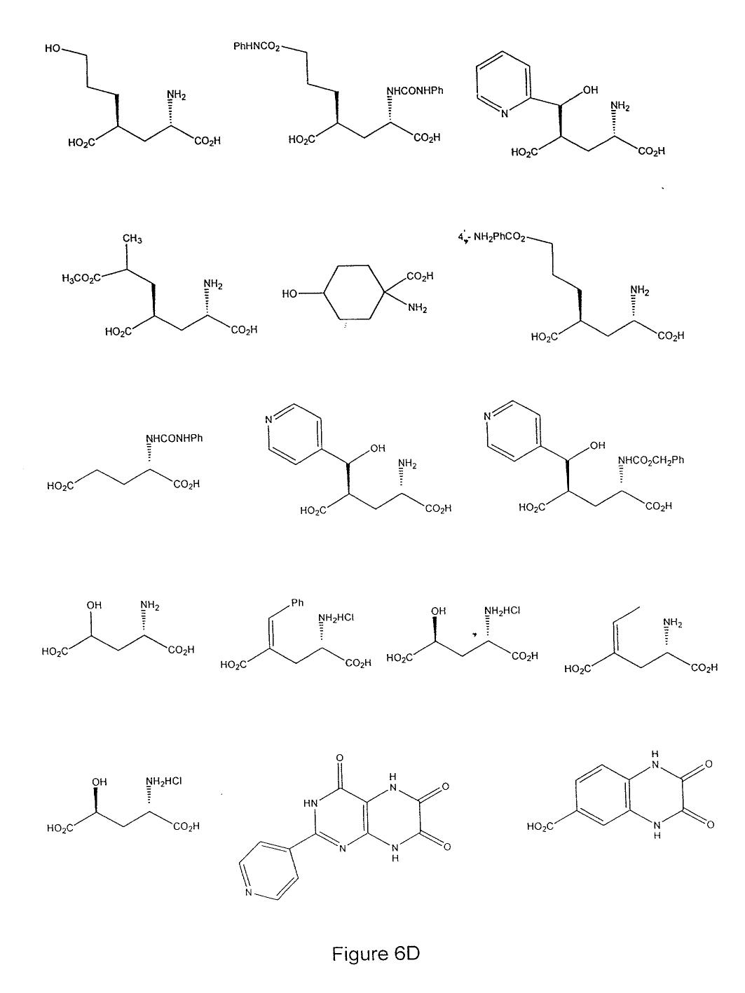

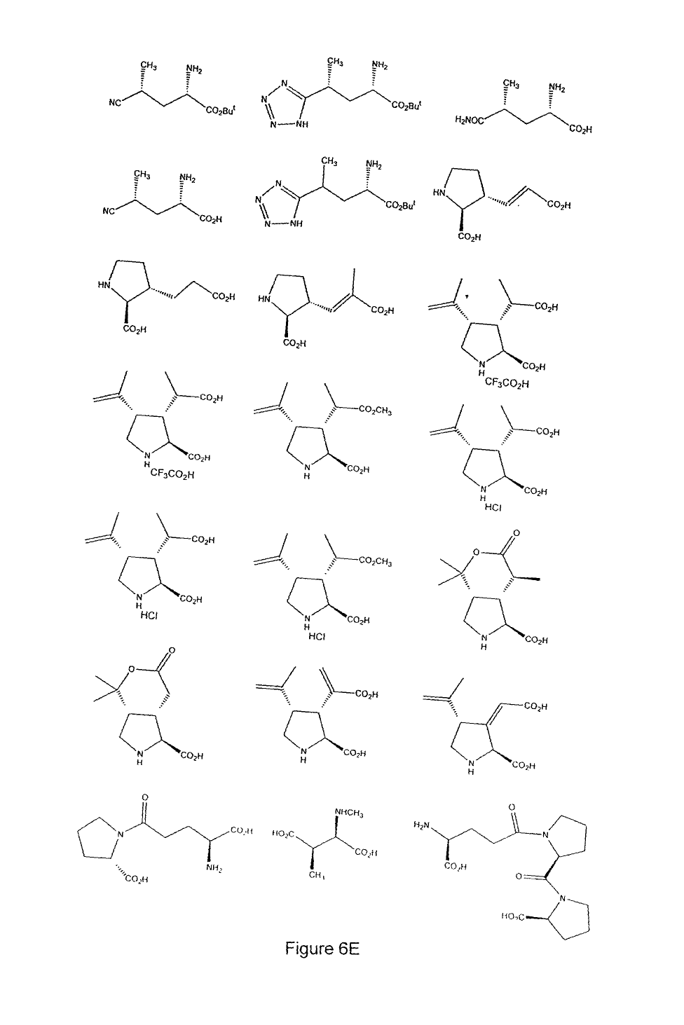

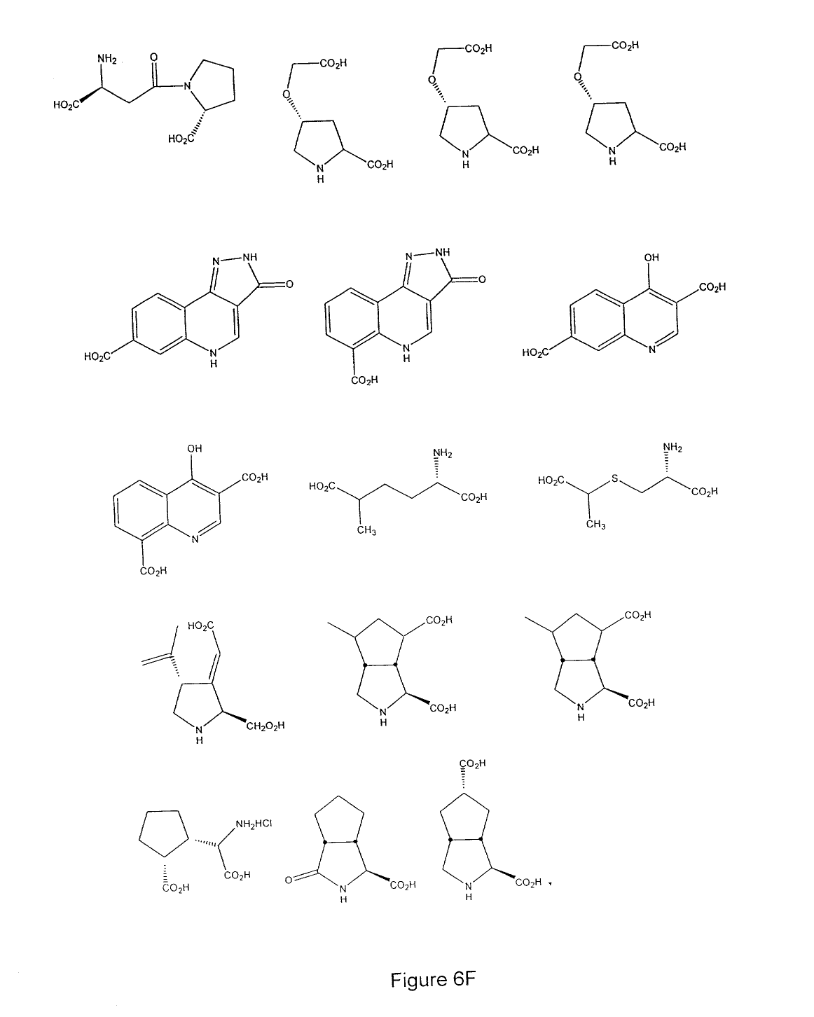

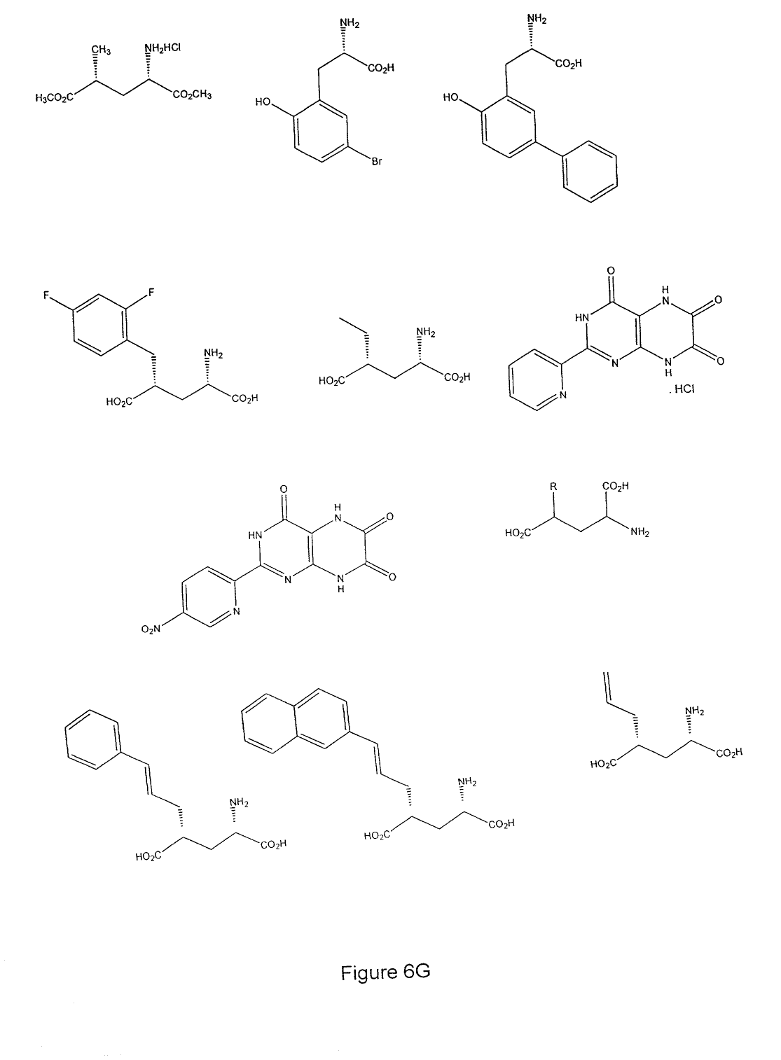

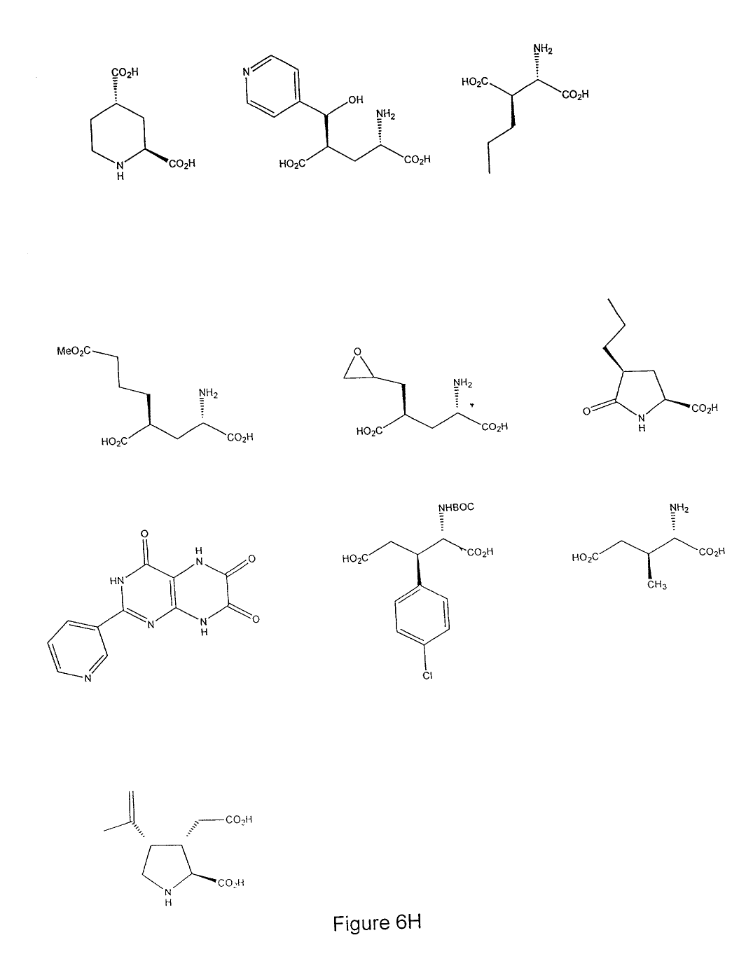

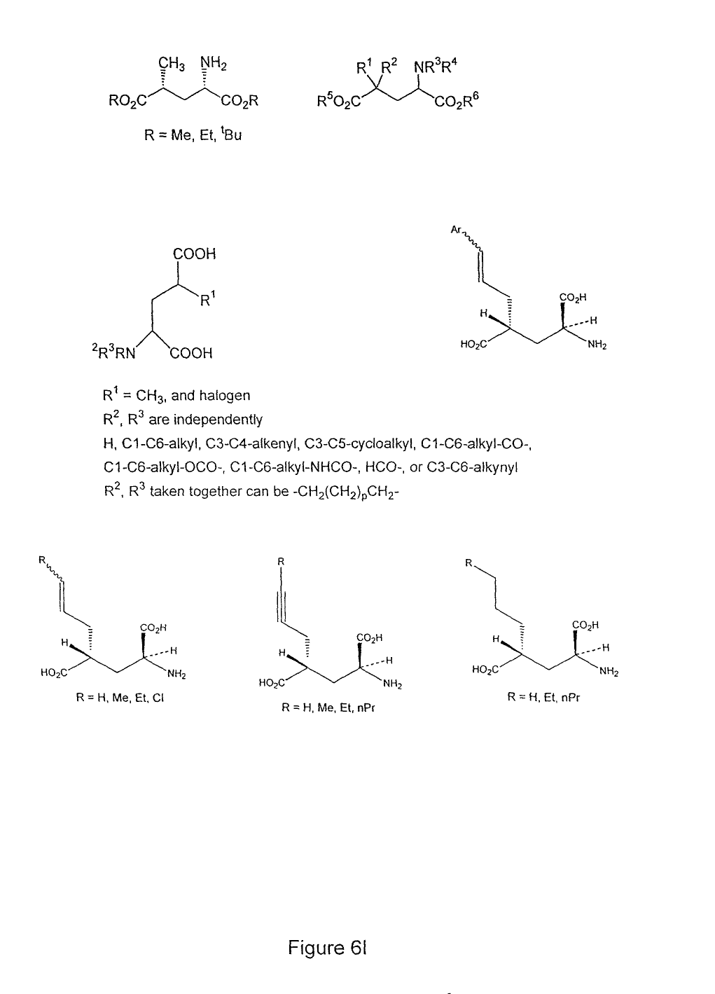

A receptor compounds is a compound that binds to one or more types of glutamate receptor. Preferred receptor compounds are compounds that are agonists or antagonists of glutamate receptors. Many such compounds are known. Examples of receptor compounds are shown in FIG. 6. The receptor compounds are used in the disclosed method to bind to glutamate transporters. By measuring the effect of test compounds on the binding of the receptor compound, compounds that bind to, and affect, the glutamate transporter can be identified. It was discovered that 4MG, which binds to glutamate receptor, can bind selectively to the glutamate transporters GLAST and GLT1. It was realized that other compounds that bind to glutamate receptors can also bind glutamate transporters.

B. Transporter Compounds

Transporter compounds are compounds identified in the disclosed assay that alter the binding of receptor compounds to glutamate transporters. Transporter compounds can be any type of molecule or conjugate including organic compounds and biological compounds. Transporter compounds can have a variety of effects on glutamate transporter activity including activation or inhibition. These compounds are expected to affect or interfere with glutamate reuptake by the glutamate transporter and thus can be used to modulate, stimulate, or inhibit glutamate reuptake. Such compounds are useful to treat various neurological diseases and conditions involving glutamate transporter and glutamate receptor activation. For example, excess extracellular glutamate is a cause of excessive activation of glutamate receptors. Stimulating glutamate reuptake by glutamate transporters can ameliorate excessive activation of glutamate receptors by reducing the extracellular glutamate concentration.

Any of the transporter compounds identified using the disclosed method can be made in prodrug form. In general, an additional moiety is added to the compound via an ester, carbonate or amine bond. such bonds can be broken in vivo resulting in generation of the active form of the compound.

C. Synthesis

The compounds to be used or screened in the disclosed method, and those identified by the disclosed method, may be prepared using synthetic reactions and techniques available in the art, as described, for example in March, “Advanced Organic Chemistry,” 4^{th} Edition, 1992, Wiley-Interscience Publication, New York. The reactions are performed in solvents suitable to the reagents and materials employed and suitable for the transformation being effected. Depending upon the synthetic route selected, and the functionality of the starting material or intermediates, the appropriate protection groups and deprotection conditions available in the art of organic synthesis may be utilized in the synthesis of the compound. It is understood by those skilled in the art of organic synthesis that the functionality present on the molecule must be consistent with the chemical transformations proposed. This will frequently necessitate judgment as to the order of synthetic steps, protecting groups required, deprotection conditions and generation of enolate to enable attachment of appropriate groups on the molecule.

D. Pharmaceutical Compositions

A. Effective Dosage Ranges

Compounds identified by the disclosed method (transporter compounds) can be administered parenterally, either subcutaneously, intramuscularly, or intravenously, or alternatively, administered orally in a dose range of between approximately 0.5 mg/kg body weight and 150 mg/kg body weight.

B. Carriers and Additives

Transporter compounds can be administered parenterally, in sterile liquid dosage forms. In general, water, a suitable oil, saline, aqueous dextrose, and related sugar solutions and glycols such as propylene glycol or polyethylene glycols are suitable carriers for parenteral solutions. Solutions for parenteral administration preferably contain a water soluble form of the active ingredient, suitable stabilizing agents, and, if necessary, buffer substances.

Antioxidizing agents such as sodium bisulfite, sodium sulfite, or ascorbic acid, either alone or in combination, can be used as suitable stabilizing agents. Also used are citric acid and its salts and sodium EDTA. In addition, parenteral solutions can contain preservatives, such as benzalkonium chloride, methyl- or propylparaben, and chlorobutanol.

Transporter compounds can be administered orally in solid dosage forms, such as capsules, tablets and powders, or in liquid dosage forms, such as elixirs, syrups, and suspensions. Gelatin capsules contain the active ingredient and powdered carriers, such as lactose, starch, cellulose derivatives, magnesium stearate, or stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere.

Other agents that can be used for delivery include liposomes, microparticles (including microspheres and microcapsules), and other release devices and forms that provide controlled, prolonged or pulsed, delivery or which enhance passage through the blood brain barrier, for example.

Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6,:275-283 (1987); and Mathiowitz, et al., J. Appl. Polymer Sci. 35:755-774 (1988). The selection of the method depends on the polymer selection, the size, external morphology, and crystallinity that is desired, as described, for example, by Mathiowitz, et al., Scanning Microscopy 4:329-340 (1990); Mathiowitz, et al., J. Appl. Polymer Sci. 45:125-134 (1992); and Benita, et al., J. Pharm. Sci. 73:1721-1724 (1984). Methods routinely used by those skilled in the art include solvent evaporation, hot melt encapsulation, solvent removal, spray drying, phase separation and ionic crosslinking of gel-type polymers such as alginate or polyphosphazines or other dicarboxylic polymers to form hydrogels.

Other delivery systems including films, coatings, pellets, slabs, and devices can be fabricated using solvent or melt casting, and extrusion, as well as standard methods for making composites.

The microparticles can be suspended in any appropriate pharmaceutical carrier, such as saline, for administration to a patient. In the most preferred embodiment, the microparticles will be stored in dry or lyophilized form until immediately before administration. They will then be suspended in sufficient solution for administration. The polymeric microparticles can be administered by injection, infusion, implantation, orally, or administration to a mucosal surface, for example, the nasal-pharyngeal region and/or lungs using an aerosol, or in a cream, ointment, spray, or other topical carrier, for example, to rectal or vaginal areas. The other devices are preferably administered by implantation in the area where release is desired. The materials can also be incorporated into an appropriate vehicle for transdermal delivery as well as stents. Appropriate vehicles include ointments, lotions, patches, and other standard delivery means.

When an alkyl substituent is identified herein, the normal alkyl structure is intended (for example, butyl is n-butyl) unless otherwise specified. However, when radicals are identified (for example, R¹), both branched and straight chains are included in the definition of alkyl, alkenyl, and alkynyl.

Pharmaceutically acceptable salts include both the metallic (inorganic) salts and organic salts; a list of which is given in Remington's Pharmaceutical Sciences 17th Edition, p. 1418 (1985). It is well known to one skilled in the art that an appropriate salt form is chosen based on physical and chemical stability, flowability, hydroscopicity and solubility.

Depending on the required activity, the disclosed compounds may be used as pharmaceutical neuroprotectants to treat acute cases of CNS injury and trauma as well as to treat convulsions, mood disorders, alleviation of pain, and other neuropsychiatric and neurodegenerative diseases due, in part, to chronic disturbances in the ion flux through glutamate receptors or in glutamate transport (such as disturbances in glutamate reuptake). Modulation of glutamate transporter activity can be used to treat both conditions resulting from a dysfunction in glutamate transport and conditions resulting from dysfunction of glutamate receptors. In the later category, for example, are conditions resulting from excessive activation of a glutamate receptor even in the presence of a physiological level of extracellular glutamate. Lowering the level of extracellular glutamate (by, for example, increasing glutamate reuptake) can reduce activation of the receptor.

Compounds identified by the disclosed method can be selected for the required activity to treat the relevant disorder. As used herein, the common definitions of neuropsychiatric and neurogenerative disorders are intended, where diagnosis is based on the alleviation of abnormal behavior, rather than histopathology.

The ester, carbonate, and amine bond in any prodrug forms of compounds identified by the disclosed method can be readily cleaved in vivo. Therefore, prodrug compositions can be hydrolyzed to the corresponding acids form in plasma. For example, (2S,4R)-4-methyl glutamic acid dimethyl ester (2) was readily hydrolyzed to generate (2S,4R)-4-methyl glutamic acid (1). In a test, 2 showed an analgesic effect similar to 1.

Method

Disclosed is a method of identifying compounds that bind to or modulate glutamate transporters. The assay involves detecting or measuring alteration of the binding of receptor compounds bound to glutamate transporters. Alteration of binding of the receptor compound is indicative of a compound that can interact with and affect the glutamate transporter. The method basically involves exposing glutamate transporters, to which a receptor compound is bound, to a test compound and detecting or measuring specific binding of the receptor compound. This is preferably done by using labeled receptor compound and following alterations in specific binding of the label. Many types and forms of labels can be used for this purpose. Preferred labels are radioactive isotopes.

The disclosed method will be specific for a subset of glutamate transporters where the receptor compound and/or conditions used result in specific binding of the receptor compound to the subset of transporters. For example, 4MG binds to only a subset of glutamate transporters (GLAST and GLT1), and so compounds that bind these receptors can be identified. Receptor compounds can also be made to selectively bind only one glutamate transporter and so compounds binding to just one of the transporters can be identified. Such specificity allows specific targeting of a glutamate transporter and thus more specific effects on the glutamate transporter by the identified compounds (that is, the identified compounds will specifically affect a specific transporter). For example, a particular condition or disease may be associated with, caused by, or affected by one of the transporters. In such a case, using a compound that specifically affects that transporter would be useful. The identified compounds can also be used to detect and assay these specific effects.

Five subtypes of glutamate transporters have been identified demonstrating discrete cellular and regional localizations as well as distinct molecular and pharmacological characteristics (Table 1).

TABLE 1

| | GLAST | GLT1 | EAAC1 | | |
|---|---|---|---|---|---|
| Transporter | (EAAT1) | (EAAT2) | (EAAT3) | EAAT4 | EAAT5 |
| Major cell type | Glia | Glia | Neurones | Neurones | ? |
| CNS distribution | Widespread | Widespread | Widespread | Cerebellum | Retina |
| Non-transportable | TBOA | MPDC, | TBOA | | THA, PDC |
| blockers | | TBOA, | | | |
| | | DHK, | | | |
| | | 3MG, KA | | | |
| Competitive | CCG-III, | CCG-III, | CCG-III, | THA, PDC, | |
| inhibitors | THA, | PDC, THA, | THA, | □AA | |
| (substrates) | MPDC, | T4HG, SOS | MPDC, | | |
| | PDC, SOS, | ACBD | PDC, SOS, | | |
| | T4HG, | | ACBD | | |
| | ACBD | | | | |

The information in Table 1 is adapted from Arriza et al., Proc. Natl. Acad. Sci. U.S.A., 94: 4155-4160 (1997); Gegelashvili and Schousboe, Mol. Pharmacol., 52: 6-15 (1997); Shimamoto et al., Mol. Pharmacol., 53: 195-201 (1998); Vandenberg, Clin. Exp. Pharmacol. Physiol., 25: 393-400 (1998); and Bridges et al., Curr. Pharmaceut. Des., 5: 363-379 (1999). AA is L-aminoadipate, CCG-III is (2S,3S,4R)-2-(carboxycyclopropyl) glycine, DHK is L-dihydrokainate, KA is kainate, 3MG is (±)-threo-3-methylglutamate, MPDC is L-anti-endo-3,4-methanopyrrolidine dicarboxylate, PDC is L-trans-pyrrolidine-2,4-dicarboxylate, SOS is L-serine-O-sulphate, T4HG is L-threo-4-hydroxyglutamate, and TBOA is DL-threo-β-hydroxyasparate.

GLAST (the rodent homologue of the human EAAT 1) is predominantly located in cerebellar Bergmann glia, but is also present in glial cells throughout the central nervous system (CNS) and transiently in a small number of neurones (Storck, et al., 1992, Proc. Natl. Acad. Sci. U.S.A. 89: 10955-10959; Furuta et al., J. Neurosci., 17: 8363-8375 (1997)). GLT1 (EAAT2) is normally present exclusively in glia, and is widespread and abundant throughout the forebrain, cerebellum and spinal cord (Pines, et al., 1992, Nature 360: 464-467; Furuta et al., 1997). The transporters EAAC1 (EAAT3) (Kanai and Hediger, 1992, Nature 360: 467-471) and EAAT4 (Fairman, et al., 1995, Nature 375: 599-603) are found predominantly in neurones, with EAAC1 being abundantly expressed throughout the brain and in spinal cord, and EAAT4 unique to Purkinje cells of the cerebellum (Furuta et al., 1997). EAAT5 is expressed predominantly in the retina, but its cellular specificity is yet to be determined (Arriza et al., 1997). The majority of synapses in the CNS are in close apposition with glial cells, and the glial transporters GLAST and GLT1 appear to be responsible for most Glu transport in the CNS (Bergles and Jahr, 1998, J Neurosci. 18: 7709-7716; Rothstein et al., 1996). Glutamate transporters are generally described in U.S. Pat. Nos. 6,020,479 and 5,739,284. EAAT1 is described in U.S. Pat. Nos. 5,919,699, 5,932,424, and 6,100,085. EAAT2 is described in U.S. Pat. Nos. 5,658,782, 5,840,516, and 5,919,628. EAAT3 is described in U.S. Pat. Nos. 5,776,774 and 6,074,828. EAAT4 is described in U.S. Pat. Nos. 5,912,171, 6,060,307, and 6,090,560. EAAT5 is described in U.S. Pat. Nos. 5,882,926 and 5,989,825.

Radiolabelled analogues of Glu, have been used (under conditions in which they do not interact with Glu receptors) to identify and characterize Glu transporters. Thus, [³H]-Glu, [³H]- L-aspartate and [³H]-D-aspartate have proved useful in biochemical and autoradiographic studies of GluTs (Li and Balcar, Exp. Brain Res., 97: 415-422 (1994); Bridges et al., 1999). The binding affinity of these radiolabelled compounds for the various GluTs does not, however, permit the study of individual subtypes of GluT.

4MG selectively binds to GLAST transporter in the presence of L-dihydrokainate (DHK) and kainate (KA) and (±)-threo-3-methylglutamate (3MG), and selectively binds to GLT1 transporter in the presence of appropriate concentrations of L-serine-O-sulphate (SOS). DHK, KA and 3MG bind to GLT1 and displaces 4MG from this transporter. DHK, KA and 3MG (at concentrations below 1 mM) do not bind GLAST leaving this transporter open for 4MG binding. SOS binds to GLAST with higher affinity than to GLT1 (Kᵢ 107 μM and 1157 μM at EAAT1 and EAAT2 respectively; Arriza, et al., 1994, J. Neurosci. 14: 5559-5569) and displaces 4MG from this transporter at concentrations that do not significantly displace binding from GLT1.

The disclosed method involves glutamate transporters which are integral membrane proteins. Thus, the disclosed method is preferably carried out using membrane preparations containing glutamate transporters. Many techniques for preparing membranes are known and can be used for the disclosed method. The method can be carried out using membranes from any tissue (from any source) that contains an appropriate glutamate transporter. Mammalian glial tissue is the preferred source of membrane preparations. Use of non-human animal tissues is preferred in view of the difficulty and expense of obtaining human tissue. However, the disclosed method can use human membrane preparations.

Binding of compounds to glutamate transporters preferably can be assayed as follows. Rat brain tissue, either freshly dissected or stored frozed at −20° C. is homogenized in HEPES buffer containing Na⁺(5 mM HEPES, 96 mM NaCl, 1.8 mM CaCl₂, 1 mM MgCl₂ (pH 7.5 at 25° C.)), and membranes are washed twice in the same buffer using standard methods for subcellular fractionation and the preparation of brain membranes. Brain tissue is weighed and homogenized in 15 ml of assay buffer using a Polytron tissue homogenizer (setting 4, 10-15 seconds). The homogenate is centrifuged (20,000 rpm/48,000 g, 15 min), the supernatant removed, and the pellet re-suspended in 15 ml fresh buffer by homogenization. The homogenate is centrifuged again (as above) and the pellet re-suspended in 20 to 25 volumes of buffer. Membrane homogenates make up 20% of final assay volume. Binding assays are also performed in the above buffer.

Binding studies are performed using a range of incubation times (1-120 min) and temperatures (4° C., 25° C., 37° C.). The range of concentrations of the receptor compound (for example, 10 nM-1 mM) is varied dependent on whether all or only a trace of the receptor compound is labeled. Following incubation of the receptor compounds (bound to the transporters) in the presence or absence of test compounds, binding is terminated by rapid filtration through Whatman GF/B filter discs and washing 3 times with buffer using a Brandel cell harvester. Binding is determined by any technique suitable for the label used. For isotope labels, scintillation spectrometry is preferred.

A range of other extensions to the disclosed method can also be used. For example, the screening procedure for glutamate reuptake inhibitors can also be performed using frozen membranes of rat brain. Those skilled in the art will recognize that the disclosed assay can be performed using of tissue from other species and other tissues in which GluTs are present, including cultured tissue (native or modified primary cell culture or cell lines). Examples of suitable sample sources are describe by Palos et al., Mol. Brain Res. 37: 297-303 (1996); Dunlop et al., Eur. J Pharmacol. 360: 249-256 (1998); Ye, et al., J. Neurosci. 19: 10767-10777 (1999); Dunlop, et al., Brain Res. 839: 235-242 (1999)). The presence of the sodium ion in the buffer is preferred. The disclosed assay can also be undertaken in buffers including but not limited to: HEPES+NaCl only, phosphate buffer (120 mM NaCl, 4.5 mM KCl, 1.2 mM CaCl₂, 1.2 mM MgCl₂, 10 mM NaHPO₄, pH 7.5 at 25° C.), Krebs phosphate buffer (118.4 mM NaCl, 4.7 mM KCl, 1.2 mM MgSO₄, 1.2 mM KH₂PO₄, 25 mM NaHCO₃, 2.5 mM CaCl₂, 11.1 mM glucose, pH 7.4 at 25° C.).

The binding assay, while preferably terminated by filtration through a commercially available cell harvester (for example, Brandel), can also be terminated by centrifugation using a commercially available microfuge according to standard methods, and those skilled in the art will recognize that the procedure can be readily adapted to robotic screening protocols.

The disclosed method is preferably performed as a high throughput screen looking at multiple test compounds simultaneously and/or sequentially. The disclosed method is suitable for automation and preferably is performed as an automated, high throughput assay.

[³H]-labeled receptor compounds can also be used for the autoradiographic localization of GluT binding in sections of rat brain tissue, and this method could be extended to include sections of other species and tissues, as described above. Following binding, sections may be apposed to tritium-sensitive film to produce images of binding localization, which may then be quantified using computer-assisted densitometry. Alternatively, sections may be wiped from slides (using filter discs or similar) and subjected to liquid scintillation counting.

Therapeutic Applications Based on In vitro and In vivo studies.

Compounds identified by the disclosed method can be used to treat a variety of diseases and conditions involving EAAs. These include cognitive, learning, and memory deficits, chemical toxicity (including substance tolerance and addiction), excitotoxicity, neurodegenerative disorder (such as Huntington's disease, Parkinson's disease, and Alzheimer's disease), post-stroke sequelae, epilepsy, seizures, mood disorders (such as bipolar disorder, dysthymia, and seasonal effective disorder), depression, and pain. Neurodegenerative disorders can result from dysfunction or malfunction of the receptor.

The disclosed method identifies compounds that bind glutamate transporters and modulate transporter function. Binding of the ultimate compounds can be determined using standard techniques. Modulation of the glutamate transporters, as demonstrated by compounds showing potent in vitro affinity for the transporter, make the compounds useful for treating human neuropsychopharmacological conditions related to EAAs. Since the compounds identified in the disclosed method regulate the in vitro transport of Glu, they are useful in the in vivo treatment of EAA-dependent psychosis, neurodegeneration, convulsions, pain, learning and memory deficits, and other conditions involving EAAs.

In combination, in vitro and in vivo assays are predictive of the activity of the identified compounds for treatment of patients. The following tests can be used to demonstrate that binding activity, consistent with the disclosed method, correlates with physiological activity, both in vitro and in vivo. The results of these tests indicate that compounds identified in the disclosed method will be effective clinically for treatment of a variety of disorders, including includes cognitive, learning, and memory deficits, chemical toxicity (including substance tolerance and addiction), excitotoxicity, neurodegenerative disorder (such as Huntington's disease, Parkinson's disease, and Alzheimer's disease), post-stroke sequelae, epilepsy, seizures, mood disorders (such as bipolar disorder, dysthymia, and seasonal effective disorder), depression, and pain. Neurodegenerative disorders can result from dysfunction or malfunction of the receptor.

A rat mechano-allodynia pain model is described by Bennett, Neuro. Report 5:1438-1440 (1994), and references cited therein. Assays useful for assessing treatment of brain and spinal cord injuries are described by Shoharni et al., J. Neurotrama 10(7):109-119 (1993), Faden, J. Neurotrama 10(7):91-100 (1993), Bruno etal., Eur. J. Pharmacology256:109-112 (1994), Long and Skolnick, Eur. J. Pharmacology 261:295-301 (1994), and Long and Skolnick, Soc. Neuroscience Abstracts 19:619.7 (1993). A Parkinson's disease model is described by Danysz et al., J. Neural Transmission 7:155-166 (1994). Assays for assessing effects on learning and memory are describe in U.S. Pat. No. 5,428,069 (column 12). An assay for assessing anxiolytic action is described by Winslow et al., Eur. J. Pharmacology 190:11-21 (1990). Assays for assessing effects on addiction are described by Papp and Willner, Psychopharmacology 103:99-102 (1991), Spyraki et al., Psychopharmacology 79:278-283 (1983), Spyraki et al., Brain Research 253:185-193 (1982), Papp et al., Eur. J. Pharmacology 317:191-196 (1996), Papp et al., J. Psychopharmacology 6(3):352-356 (1992), Przegalinski et al., Pharmacology Biochemistry Behavior 54(1):73-77 (1996), Evoniuk et al., Psychopharmacology 105:125-128 (1991), and Kolesnikov et al., Life Sciences 55(18):1393-1398 (1994). Assays for assessing effects on depression are described by Przegalinski et al., Neuropharnacology 36(1):31-37 (1997), and Skolnick et al., Pharmacopsychiat. 26:23-26 (1996).

The following specific examples of assays and models can be used to assess the activity of compounds identified using the disclosed method.

Neurodegenerative Transient Global Forebrain Ischemia

The extent of protection by a test compound in a model of brain ischemia can be assayed as described in Meldrum et al., Brain Res., 571:115, 1992, and references cited therein. Male Wistar rats (250-300 g) are anesthetized using halothane-oxygen-nitrogen mixture and both vertebral arteries are permanently occluded by electrocauterisation within the alar foraminae of the first cervical vertebra. At the same time, both common carotid arteries are isolated and atraumatic clamps placed around each one. One femoral vein is cannulated to enable the subsequent iv administration of fluid. The following day cerebral ischemia is induced in the unanaesthetised animal, by tightening the clamps around the carotid arteries for 20 min. Carotid clamping results. Body temperature is maintained at 37° C. by use of a rectal probe and hot plate. Seven days after the ischemic insult rats are sacrificed and the brains processed for light microscopy. Neuroprotection is assessed by examination of the extent of damage in the cortex and hippocampus. Compounds may be selected which are active in this model.

Neurodegenerative Permanent Focal Ischemia

The extent of protection by a test compound in a model of brain ischemia can be assayed as described by Meldrum and Smith (Stroke, 23:861, 1992), and references cited therein. Male Fisher F344 rats (210-310 g) are anesthetized with halothane-oxygen-nitrogen mixture receive a small incision between the eye and ear, the mandibular muscles are retracted to expose the orbit and zygomatic arch. A small craniotomy is made to expose the base of the middle cerebral artery. Bipolar coagulation is used to permanently occlude the artery at the base. One day after the ischemic insult rats are sacrificed and the brains processed for light microscopic examination. Lesion volume is determined by using Cavalarei's principle. Compounds may be selected which are active in this model.

Maximum Electro Shock (MES) Seizure Test

This test is to determine the extent of protection by a test compound in a seizure model. This model is described by Rogawski et al. (Epilepsy Research, 15:179-184, 1993). Male NIH Swiss mice (25-30 g) were injected ip with the test drug. The mice were subjected to a 0.2 sec, 60 Hz, 50 mA electrical stimulus delivered with corneal electrodes wetted with 0.9% saline at 15-30 min post dosing. Animals failing to show tonic hind limb extension were scored as protected. Compounds may be selected which are active in this model.

Subcutaneous Metrazol (scMET) Seizure Test

This test is to determine the extent of protection by a test compound in a seizure model. The method used is that of Chen et al. (Proc. Soc. Exp. Biol. Med, 87:334, 1954). Mice are randomly assigned to vehicle or treatment groups of 3-10 animals per group and then dosed accordingly. Metrazol (pentylenetetrazol) 90 mg/kg is administered subcutaneously (sc) at different time points (0.25, 0.5, 1, 2, 4 hr) after the treatment or control groups. The mice individually housed in clear runs and observed for the presence or absence of clonic seizure activity (>5 s duration) for 30 min after metrazol dosing. A compound is considered active if no seizure is observed. Data is analyzed using a quantal measure (protection/number tested).

Rat Mechano-allodynia Pain Model

This test is to determine the extent of protection by a test compound to neuropathic pain sensations. The model is described by Bennett, Neuro. Report 5:1438-1440 (1994), and references cited therein. A rat is prepared by bilaterally exposing the sciatic nerves on both thighs. On one side, loosely fitting constrictive ligatures are tied around the nerve; the other side is sham manipulated but ligated. With the rat standing on an elevated perforated floor, mechano-allodynia is measured by applying from beneath a graded series of yon Frey hairs to the mid-plantar region of the effected paws. The hair that evokes at least one withdrawal response is designated the threshold level when compared to the sham treated nerve.

Mouse Antidepressant Forced Swim Test

This test is to determine the extent of antidepressant activity of a test compound. The model described by Trullas et al., Eur. J. Pharm. 185, 1-10 (1990), and the references cited therein. Mice are placed individually in a cylinder filled with water at 22° C.-25° C. The duration of immobility is scored during the last four minutes of a six minute test.

Elevated Plus Maze

Compounds with antidepressant activity increase both the percentage of time and percentage of entries into the open arms of an elevated plus-maze as described by Trullos et al., “1 -Aminocyclopropanecarboxylates exhibit antidepressant and anxiolytic actions in animal models,” Eur. J. Pharm., 203:379-385 (1991). A mouse is placed at the intersection of the maze arms so that its head is in the center of the platform. The mouse is then scored as being in the open or enclosed arms. Arm entries are recorded and the percentage of time in each arm, as well as the percentage of entries, are calculated. The compounds are expected to be active in this test at a dose of about 1-40 mg/kg ip.

NMDA Induced Seizures

Compounds which have anticonvulsant activity for convulsions are active in a test described by Koek et al., Mechanisms for Neuromodulation and Neuroprotection, pp 665-671, Kamenka et al., eds., NPP Books, Ann Arbor, Mich. 1992. Test compounds are injected into mice at 15 minutes or 30 minutes before an ip injection of NMDA, icv or ip, respectively. ED₅₀ is determined by comparing the percentage of mice that die after 30 minutes to a group of mice that receive NMDA alone. The compounds are expected to be active in this test at a dose of about 1-40 μg/kg icv, or 1-40 mg/kg ip.

Cocaine-induced Hypermotility

Kainate administered locally or cocaine administered subcutaneous (s.c.) induces an increase in dopamine release in nucleus accumbens and nucleus caudatus accompanied by stereotyped behavior such as hyper-locomotion, rearing, sniffing, and grooming. Inhibition of these effects can be used to indicate that a compound useful for treating addiction.

Cocaine Induced Convulsions

Compounds with anticonvulsant activity for cocaine induced convulsions are active in tests described by Witkin et al., Life Sciences, 48:51-56, 1991. Male Swiss Webster mice, 10-12 weeks old, are injected with the test compound ip 30 minutes prior to an ip injection of 75 mg/kg cocaine. The occurrence of convulsions is recorded for 15 minutes following the cocaine injection and are defined as loss of righting responses for at least 5 seconds and the occurrence of clonic limb movement. The ED₅₀ dose can then be calculated. The compounds are expected to be active in this test at doses of about 1-40 mg/mg ip.

### EXAMPLES

### Example 1

### Synthesis and use of [³H]-(2S,4R)-4-methylglutamate ([³H]-4MG)

A. Synthesis

A racemic mixture of (2S,4S), (2R,4R), (2S,4R), and (2R,4S)-4-methyl glutamic acid was prepared as follows. Sodium metal in small pieces was dissolved in absolute ethanol, and to this solution sulfur and diethyl acetamidomalonate are added. Then methyl methacrylate was added dropwise while refluxing and the reaction mixture refluxed further. The solvent was removed by evaporation in vacuo and the residue crystallized from ethanol. This solid material was refluxed in HCl then the solvent removed by evaporation in vacuo. The residue was dissolved in distilled water and washed with trioctylamine in CHCl₃. The aqueous phase was evaporated in vacuo and the oily residue dissolved in a very small amount of water and crystallized with an excess of acetone to result in 4-methyl glutamic acid. (2R,4S) and (2S,4R)-4-methyl glutamic acid was obtained by fractional crystallization in water and acetone at 4° C. from racemic diastereomers obtained above.

[³H]-(2S,4R)-4-methylglutamate was directly synthesized as follows. (S)-1-t-Butoxycarbonyl-5-t-butyldiphenylsiloxymethyl-pyrrolidine-2-one was prepared using literature procedures from (S)-(−)-2-pyrrolididone-5-carboxylic acid. To a solution of (S)-1-t-butoxycarbonyl-5-t-butyldiphenylsiloxymethyl-pyrrolidine-2-one in THF at −78° C. a 1 M solution of LiN(SiMe₃)₂ was slowly added. After stirring for 1 hour, iodomethane was added. The reaction mixture was allowed to stir for another 2 hour at −78° C. and then quenched with acetic acid. The mixture was concentrated into half volume, diluted with water and extracted with ethyl acetate (EtOAc). The combined extracts was washed with water, brine and dried over MgSO4. The solvent was evaporated in vacuo and the residue purified by flash column chromatography on silica gel, eluted with EtOAc:Hexane (1:2.5). The desired (2S,5R) isomer ((3R,5S)-1-t-Butoxycarbonyl-5-t-butyldiphenylsiloxymethyl-3-methyl-pyrrolidine-2-one) was eluted first from the column and crystallized in hexanes to give the product.

To a solution of (3R,5S)-1-t-butoxycarbonyl-5-t-butyldiphenylsiloxymethyl-3-methyl-pyrrolidine-2-one and glacial acetic acid in dry THF at 0° C. was added a 1.0 M solution of tetrabutylammonium fluoride in THF. The solution was allowed to warn room temperature and stir overnight. Ethyl acetate was added and the organic phase extracted with aqueous ammonium chloride. The combined aqueous phases was extracted with ethyl acetate. The combined organic phases was washed with brine, dried over Na₂SO₄, filtered and evaporated to dryness. The crude product (principally (3R,5S)-1-t-Butoxycarbonyl-5- hydroxymethyl-3-methylpyrrolidine-2-one) is purified by filtration through silica gel, eluted with EtOAc in hexane to give the product as a clear oil, which was carried on for next step without further characterization.

To a solution of (3R,5S)-1-t-butoxycarbonyl-5-hydroxymethyl-3-methylpyrrolidine-2-one in a solvent mixture of acetonitrile: carbon tetrachloride: water (2:2:3) was added sodium periodate and ruthenium trichloride. The solution was stirred 2 hours at room temperature and then diluted by the addition of dichloromethane and brine. The organic phase was separated and the aqueous phase was extracted with dichloromethane. The combined organic phases were dried over Na₂SO₄ then celite added. The solution was filtered under suction through a bed of celite and the filtrate was evaporated to give an oily residue from which (3R,5S)-1-t-Butoxycarbonyl-5-carboxy-3-methylpyrrolidine-2-one was crystallized in ethyl acetate and hexane. The mother liquor was purified by flash column chromatography on silica gel, eluting with ethyl acetate in hexane to give additional product.

A solution of (3R,5S)-l-t-Butoxycarbonyl-5-carboxy-3-methylpyrrolidine-2-one (6.2 g, 25.5 mmol) in THF (50 mL) was treated with lithium hydroxide monohydrate and water. After stirring for 16 hours at room temperature the THF was removed in vacuo and water was added. The pH was adjusted to 3 by the addition of glacial acetic acid, ether was added, and the layers was separated. The aqueous phase was extracted with ether and the combined organic phases were washed with brine, dried over Na₂SO₄, filtered and evaporated to dryness, azeotroping with toluene. The residue was dried under high vacuum to give (2S,4R)-N-t-Butoxycarbonyl-4-methyl glutarnic acid as a white foam which can be used without further purification.

The Boc-protected diacid, (2S, 4R)-N-t-butoxycarbonyl-4-methyl glutamic acid, was subjected to a mixture of trifluoroacetic acid: methylene chloride (40:60) for 3 hours at room temperature. The volatiles were removed in vacuo and the residue was azeotroped with toluene. Water was added and the aqueous phase extracted with a 5% solution of trioctylamine in chloroform. The combined organic phases are washed with water and the combined aqueous phases evaporated by being placed on a lyopholyzer for 48 hours to give [3H]-(2S,4R)-4-methyl glutamic acid as a white foam which was recrystallized in acetone and water.

### Example 2

### Binding of 4MG to Brain Tissue

The binding of 4MG to brain tissue was demonstrated and the binding characteristics of 4MG to glutamate transporters were analyzed. Forebrain tissue from female Sprague Dawley rats was homogenised in HEPES buffer containing Na⁺, and membranes were washed twice in the same buffer. Binding experiments were also performed in this buffer. Initial studies determined appropriate parameters for time (15 minutes incubation at 4° C.) and drug concentration (20 nM [³H]-4MG) to be used in subsequent experiments. Following incubation of [³H]-4MG with or without other compounds, binding was terminated by rapid filtration through Whatman GF/B filter discs and washing 3 times with buffer using a Brandel cell harvester. Binding was determined by scintillation spectrometry. A full range of association, dissociation, saturation and inhibition studies was performed over varying incubation times (up to 60 min). Data were analyzed using computer-assisted curve fitting. Values are expressed as mean of at least triplicate determinations and multiple experiments.

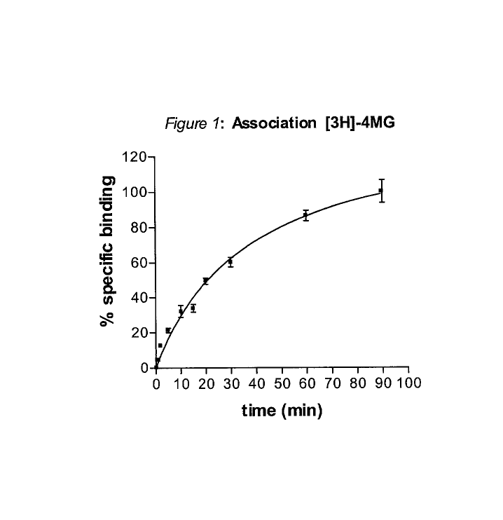

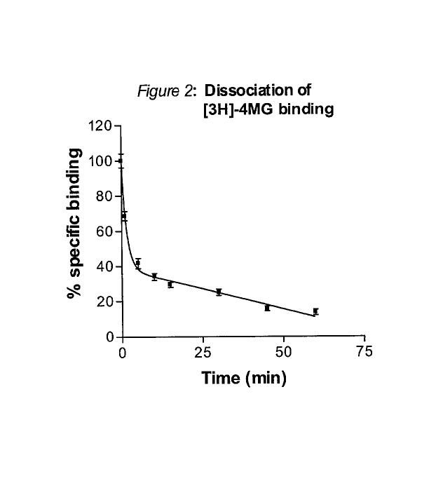

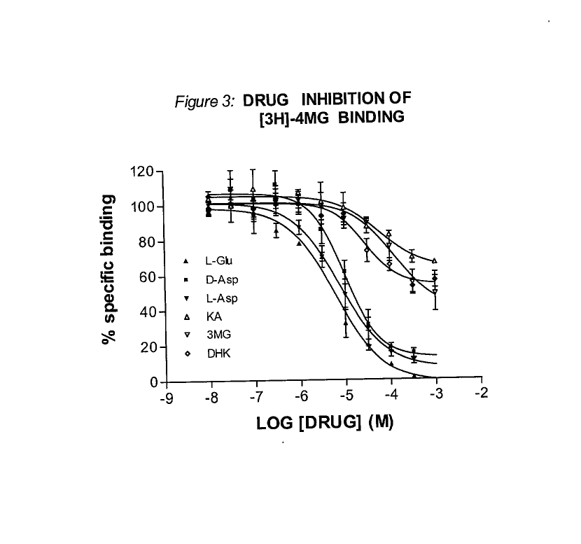

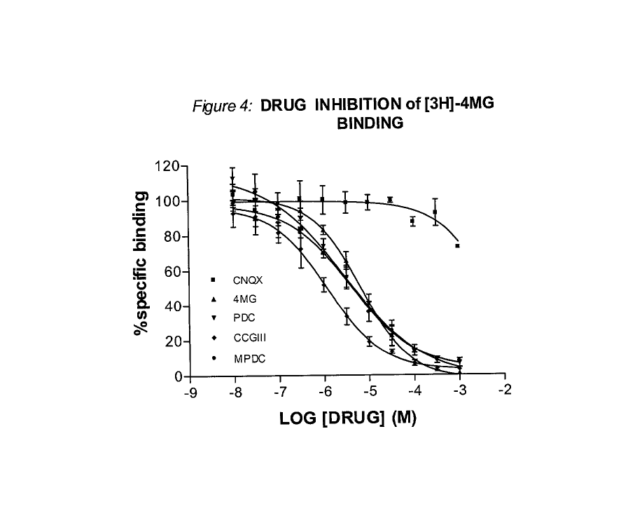

4MG was shown to bind specifically to rat brain membrane homogenates. Specific binding of 4MG (20 nM) to rat brain membrane homogenates at 4° C. represented greater than 90% of total binding (non-specific binding defined using 1 mM Glu). 4MG binding was rapid and saturable, with an association t_{½} of approximately 20 min (K_{D}=5.4 μM, Bₘₐₓ=30 pmol/mg protein; FIG. 1) and a dissociation t_{½} of approximately 3 min (FIG. 2). This binding could be completely displaced by the EAAs glutamate (IC₅₀6.1 μM), L-aspartate (IC₅₀ 7.5 μM), and D-aspartate (IC₅₀10.9 μM) (FIG. 3), and by the glutamate transporter inhibitors (2S,3S,4R)-2-(carboxycyclopropyl)glycine (CCG-III; IC₅₀ 1.2 μM), L-trans-pyrrolidine-2,4-dicarboxylate (PDC; IC₅₀ 2.9 μM), L-anti-endo-3,4-methanopyrrolidine dicarboxylate (MPDC; IC₅₀4.0 μM), and 4MG (IC₅₀6.7 μM) (FIG. 4).

Specific compounds may be used to block one of the two glial components of the binding of [³H]-4MG, allowing the study of these individual transporters (GLAST or GLT1) in isolation. L-Dihydrokainate (DHK), kainate (KA) and (±)-threo-3-methylglutamate (3MG) are known to specifically bind to only GLT1 (EAAT2) at certain concentrations, and in our experiments these compounds displace some 35-50% of [³H]-4MG binding. L-serine-O-sulphate (SOS) has reported Kᵢ values of 107 μM and 1157 μM at EAAT1 and EAAT2 respectively when these GluTs are expressed in cells of the COS-7 line (Arriza et al., 1994, J. Neurosci. 14: 5559-5569] and Kₘ values of 39 and 240 μM respectively when expressed in Xenopus oocytes (Vandenberg et al., 1998, Br. J. Pharmacol. 123: 1593-1600). SOS exhibited an IC₅₀ of 40 μM for binding of [³H]-4MG, indicating that this compound is suitable for displacing [³H]-4MG from GLAST (EAAT1), thus allowing specific binding of [³H]-4MG to GLT1 (EAAT2).

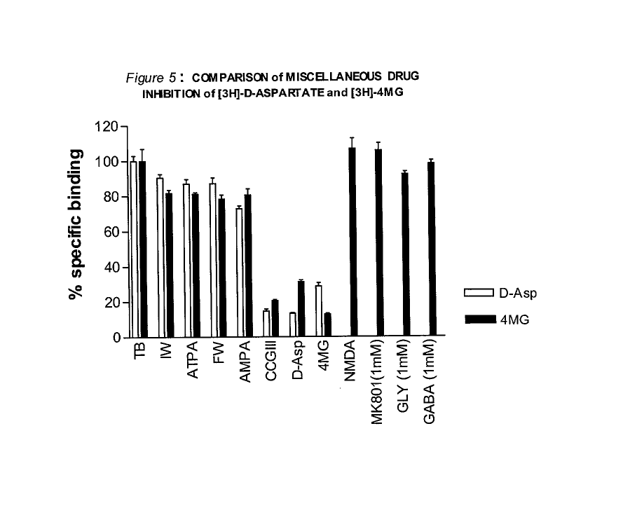

The low affinity kainate receptor antagonist 6-cyano-7-nitroquinoxaline (CNQX) did not significantly displace binding of 4MG at 100 μM (FIG. 4). In addition, agonists and antagonists at other subtypes of Glu receptor (N-methyl-D-aspartate (NMDA), (+)-5-methyl- 10,11 -dihydro-5H-dibenzo[a,d]cyclohepten-5,10-imine maleate (MK801), α-amino-3-hydroxy-5-methyl-4-isoxazolepropionate (AMPA), and fluorowillardiine (FW)) also failed to significantly displace 4MG binding at or above 100 μM (FIG. 5). The low affinity KA agonists ATPA and iodowillardine (IW; see Carroll et al., 1998), and the inhibitory amino acid neurotransmitters, glycine (GLY) and γ-aminobutyric acid (GABA) also failed to affect 4MG and/or D-aspartate binding to GluTs (FIG. 5).

A full range of association, dissociation, saturation and inhibition studies was performed over varying incubation times (up to 60 min). Data were analyzed using computer-assisted curve fitting. Values cited are expressed as mean of at least triplicate determinations and multiple experiments.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A method of identifying compounds that bind to or modulate glutamate transporters, the method comprising
bringing into contact a test compound and a glutamate transporter bound by a receptor compound, and
detecting alteration of binding of the receptor compound bound to the glutamate transporter,
wherein alteration of binding of the receptor compound identifies the test compound as one that binds to or modulates the glutamate transporter.

2. The method of claim 1 wherein the receptor compound is an agonist of a glutamate receptor.

3. The method of claim 1 wherein the receptor compound is an antagonist of a glutamate receptor.

4. The method of claim 1 wherein the receptor compound is a ligand of a glutamate receptor.

5. The method of claim 1 wherein the receptor compound is selectively bound to one type of glutamate transporter.

6. The method of claims 5 wherein the glutamate transporter is GLAST, GLT1, EAAT1, or EAAT2.

7. The method of claim 5 wherein the receptor compound is bound to the glutamate transporter in the presence of a compound with appropriate selectivity.

8. The method of claim 5 wherein the receptor compound is bound to the glutamate transporter in the presence of L-dihydrokainate or L-serine-O-sulphate.

9. The method of claim 1 wherein the receptor compound is bound to the glutamate transporter in the presence of sodium ion.

10. The method of claim 1 wherein the method is performed on a plurality of test compounds.

11. The method of claim 10 wherein the method is automated.

12. The method of claim 10 wherein the method is performed on a plurality of test compounds simultaneously, sequentially, or a combination.

13. The method of claim 1 wherein the receptor compound has the structure 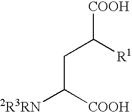
wherein R¹=CH₃ or halogen,
R² and R³ are independently
H, C1-C6-alkyl, C3-C4-alkenyl, C3-C5-cycloalkyl, C1-C6-alkyl-CO—, C1-C6-alkyl-OCO—, C1-C6-alkyl-NHCO—, HCO—, or C3-C6-alkynyl R² and R³ taken together can be —CH2(CH2)pCH2—

14. The method of claim 1 wherein the receptor compound has the structure 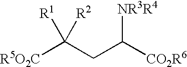
wherein
R¹, R², R⁵ and R⁶ are independently
1) C1-C6-alkyl,
2) C3-C4-alkenyl,
3) C3-C5-cycloalkyl;
4) H;
R³ and R⁴ are independently
1) H
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) HCO—, or
9) C3-C6-alkynyl;
R³ and R⁴ taken together can be —CH₂(CH₂)ₙCH_{2—};
n is 0-3.

15. The method of claim 1 wherein the receptor compound has the structure 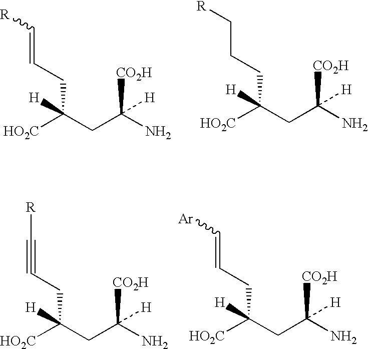
wherein R=H, C1-C6-alkyl, C3-C4-alkenyl, C3-C5-cycloalkyl, C1-C6-alkyl-CO-, C1-C6-alkyl-OCO-, C1-C6-alkyl-NHCO-, HCO-, or C3-C6-alkynyl.

16. The method of claim 1 wherein the receptor compound has one of the structures as shown in FIGS. 6A-I.

17. The method of claim 13 wherein the receptor compound is labeled with at least one [³H].

18. The method of claim 17 wherein the receptor compound is [3H]-(2S,4R)-4-methylglutamate.

19. The method of claim 5 wherein the receptor compound is [³H]-(2S,4R)-4-methylglutamate.

20. The method of claim 6 wherein the receptor compound is [³H]-(2S,4R)-4-methylglutamate.

21. The method of claim 7 wherein the receptor compound is [³H]-(2S,4R)-4-methylglutamate.

22. The method of claim 8 wherein the receptor compound is [³H]-(2S,4R)-4-methylglutamate.

23. The method of claim 9 wherein the receptor compound is [³H]-(2S,4R)-4-methylglutamate.

24. The method of claim 17 wherein the method is automated.

25. A compound obtained by the of claim 1 wherein the compound binds to or modulates the glutamate transporter.

26. The compound of claim 25 obtained by the method of claim 18.

27. A method of using the compound of claim 25 in medicine comprising administering to a mammalian subject a pharmaceutical composition which comprises the compound.
